(19) United States
(12) Patent Application Publication
(10) Pub. No.: US 2022/0187310 A1
(43) Pub. Date: Jun. 16, 2022

(54) ANTI-DRUG ANTIBODY ASSAY

(71) Applicants: Adverum Biotechnologies, Inc., Menlo Park, CA (US); Charles River, Worcester, MA (US)

(72) Inventors: Judith GREENGARD, Menlo Park, CA (US); Mark RENZ, Menlo Park, CA (US); Valerie THEOBALD, Worcester, MA (US)

(21) Appl. No.: 17/685,705

(22) Filed: Mar. 3, 2022

Related U.S. Application Data
(63) Continuation of application No. PCT/US2020/049352, filed on Sep. 4, 2020.
(60) Provisional application No. 62/896,361, filed on Sep. 5, 2019.
(60) Provisional application No. 62/928,567, filed on Oct. 31, 2019.

(51) Int. Cl. G01N 33/68; G01N 33/543; G01N 33/553

(52) U.S. Cl. CPC G01N 33/6854; G01N 33/553; G01N 33/54306

(57) ABSTRACT

Provided is an immunoassay for detection of anti-drug antibodies (ADAs), such as anti-C1-Inhibitor antibodies (C1INH-ADA in a test sample. The immunoassay provides a means for testing the immunogenicity and efficacy of drug treatment protocols.

### CROSS REFERENCE TO RELATED APPLICATIONS

This application is a continuation of and claims the benefit of and priority to PCT Application US2020/049352, titled “Anti-Drug Antibody Assay” filed Sep. 4, 2020, U.S. Provisional Application No. 62/896,361, filed Sep. 5, 2019 and U.S. Provisional Application No. 62/928,567, filed Oct. 31, 2019, the entire contents of which being incorporated herein by reference in their entireties for all purposes.

### TECHNICAL FIELD

The present disclosure relates to an immunoassay for detection of anti-drug antibodies (ADAs), such as anti-C1-Inhibitor antibodies (C1INH-ADA) in a test sample. The immunoassay comprises the steps of (i) acidic dissociation of the anti-drug antibodies from the drug within a provided test sample; (ii) neutralization of the test sample containing the dissociated drug-ADA complexes; (iii) incubation of the sample with excess binding affinity-labeled drug; (iv) capture of the resulting ADA-binding affinity labeled drug complexes on an affinity binding substrate surface; (v) addition of tagged anti-human antibodies; and (vi) quantification of captured affinity-labeled drug-ADA complexes. The immunoassay provides a means for testing the immunogenicity and efficacy of drug treatment protocols.

### BACKGROUND

Hereditary angioedema (HAE) is a rare disorder that is associated with excess bradykinin generation resulting from a deficiency of C1-Inhibitor (C1INH). C1INH is a serpin important in control of plasma serine proteases that release bradykinin from high molecular weight kininogen. Excess bradykinin within a subject can lead to swelling which can be life threatening, especially when it occurs in the airways.

Several protein replacement therapies are currently on the market for treatment of HAE. These include, for example, administration of recombinant C1INH (Haegarda®, Ruconest®, Berinert®, C1INRYZE®). Methods of treating HAE using gene therapy have been proposed, whereby a functional copy of C1INH would be delivered to a subset of cells of a patient with HAE, thereby producing sufficient HAE to reduce or alleviate symptoms. See WO2016/191746. Unwanted immunogenicity is an immune response sometimes observed with administration of a therapeutic protein leading to production of anti-drug-antibodies (ADAs) which can inactivate the therapeutic effects of the treatment and, in some cases, inducing adverse effects. The development of ADAs to a gene therapy expression product is a concern because expression of the therapeutic protein is prolonged or permanent.

Clinical trials have reported low incidence of anti-drug antibodies (ADA) to C1INH in treated subjects; however, the drug tolerance of these assays has not been described. High levels of soluble analyte are widely known for interference in ADA assays; normal serum concentrations of C1INH range from 180-200 μg/mL (1.7-2 μM). Post-marketing commitments for C1INH replacement therapies will include a requirement to develop and validate a well-controlled and sensitive assay for C1INH immunogenicity. Accordingly, highly drug-tolerant assays for ADA to C1INH in human test samples are needed.

### SUMMARY

For regulatory purposes, it is advantageous to be able to detect and quantify the development in a subject of anti-drug antibodies against a drug treatment.

The present disclosure relates to an immunoassay for detection of the presence of anti-drug antibodies (ADA) in a sample. The immunoassay comprises the steps of (i) providing a test sample with an acidic environment for dissociation of anti-drug antibodies from the drug within a provided test sample; (ii) neutralization of the test sample containing the dissociated drug-ADA complexes; (iii) incubation of the sample with excess binding affinity-labeled drug; (iv) capture of the resulting ADA-binding affinity labeled drug complexes within the test sample on an affinity binding substrate surface; (v) addition of tagged anti-human antibodies; and (vi) quantification of captured antibody-drug-ADA complexes.

The present disclosure relates to an anti-drug antibody immunoassay for detection of the presence of anti-C1-Inhibitor antibodies (C1INH-ADA) in a sample. The assay comprises the steps of (i) providing a test sample with an acidic environment for the dissociation of C1INH-ADA complexes within the provided test sample; (ii) neutralization of the test sample containing the dissociated C1INH-ADA complexes; (iii) incubation of the test sample with excess of affinity-labeled C1INH resulting in formation of labeled C1INH-ADA complexes; (iv) capture of the resulting labeled C1INH-ADA complexes within the test sample on a functionalized substrate surface; (v) addition of tagged anti-human immunoglobulin secondary antibodies that bind to the captured labeled C1INH-ADA complexes; and (vi) quantification of captured labeled C1INH-ADA complexes.

The present disclosure relates, more specifically, to an anti-drug antibody immunoassay for detection of the presence of anti-C1-Inhibitor antibodies (C1INH-ADA) in a sample. The assay comprises the steps of (i) providing a test sample with an acidic environment for the dissociation of C1INH-ADA complexes within the provided test sample; (ii) neutralization of the test sample containing the dissociated C1INH-ADA complexes; (iii) incubation of the test sample with excess biotin-labeled C1INH resulting in formation of biotin-labeled C1INH-ADA complexes; (iv) capture of the resulting biotin-labeled C1INH-ADA complexes within the test sample on a functionalized streptavidin substrate surface; (v) addition of tagged anti-human immunoglobulin secondary antibodies that bind to the streptavidin captured biotin-labeled C1INH-ADA complexes; and (vi) quantification of captured biotin-labeled C1INH-ADA complexes.

A test sample to be measured refers to a sample possibly containing drug-ADA complexes and, for example, is a sample collected from a subject being treated with a given drug. In other embodiments, the sample is obtained from a subject who has not recently been exposed to the drug or obtained from the subject prior to the planned administration of the drug. A subject may be a mammal, for example a human, with a disease or suspected of having a disease for which drug treatment is, or will be, administered. However, in some instances, the term “subject”, as used herein, refers to laboratory animal of an animal model study. In embodiments the sample is, or can be derived from, a bodily fluid or body tissue. A test sample may comprise a material selected from the group consisting of body fluids, blood, whole blood, plasma, serum, mucus secretions, saliva, lymph fluid or an immunoglobulin-enriched fraction derived from one or more of these tissues.

In a specific step of the disclosed immunoassay, the test sample suspected of having ADAs is exposed to an acidic environment to dissociate drug-ADA complexes within the sample. The acidic environment is one that is sufficiently acidic to result in dissociation of the drug-ADA complex of interest and can be determined by one of skill in the art. Typically, such an acidic environment is in the pH range of pH 2.0 to pH 5.0, preferably pH 2.0 to pH 3.0, preferably pH 2.6. The sample is then neutralized and labeled C1INH is added at the same time to the reaction.

In certain aspects, affinity-binding pairs comprising a first member of a binding pair and a second member of a binding pair are used for capture of drug-ADA complexes on a substrate surface. In such affinity binding pairs, the first member of the binding pair has binding affinity for the second member of a binding pair. Such affinity binding pairs may be selected, for example, from the group consisting biotin/streptavidin, biotin/avidin, GST/glutathione, His-tag/Nickel, calmodulin binding protein/calmodulin, maltose binding protein/maltose, enzyme-enzyme substrate, and receptor-ligand binding pairs.

In an embodiment, the binding affinity label associated with the drug comprises a first member of a binding pair and the affinity binding substrate surface for use in capture of the labeled drug-ADA complex comprises a second member of a binding pair. In a specific embodiment, the affinity binding pair comprises a biotin/streptavidin binding pair. In such an instance, the drug, e.g., C1INH, is labeled with biotin and the binding substrate surface comprises streptavidin molecules to which the biotin binds. In one aspect, the binding substrate surface is Meso Scale Discovery (MSD)-Gold streptavidin-coated plates.

For detection of drug-ADA complexes bound to the binding substrate surface of the assay, tagged anti-human secondary immunoglobulin antibodies are used in the practice of the assay. A secondary antibody is an antibody which binds to other antibodies, for example, a mouse antibody which binds human antibodies (a mouse anti-human immunoglobulin secondary antibody). Such anti-human secondary antibodies include anti-human immunoglobulin antibodies that are labeled with a phosphorescent moiety, luminescent moiety, electrochemiluminescent moiety, chromatic moiety, a radioactive isotope or an enzyme. In one embodiment, the detectable label comprises an electrochemiluminescent label comprising a sulfo-TAG.

The present disclosure further provides kits that are assembled for determining the presence or absence of ADAs in a test sample. The kits may comprise instructions and, in a container, reagents including (i) for contacting the sample with an acid solution; (ii) for neutralization of the sample. The kit will further comprise one or more of the following reagents (i) a binding affinity labeled drug; (ii) an affinity binding substrate for capture of ADA-binding affinity labeled drug complexes; and tagged anti-human secondary antibodies.

### BRIEF DESCRIPTION OF THE DRAWINGS

Various embodiment of the peptide compositions and methods are described herein with reference to the drawings wherein:

FIG. 1 is a schematic of the anti-drug antibody assay for C1 Esterase Inhibitor.

FIG. 2 illustrates the steps of the anti-drug antibody assay.

FIG. 3 is a graph depicting screening cut points. The observed ECL values were analyzed by two operators over four runs.

FIG. 4 is a graph depicting confirmatory cut points. The observed percentage inhibition was analyzed by two operators over four runs.

FIG. 5 shows electrochemiluminescence (ECL) values in unspiked sera. ECL values are plotted against different test human plasmas.

FIG. 6 shows electrochemiluminescence (ECL) values in low positive control (LPC) (75 ng/mL). ECL values are plotted against different test human plasmas.

FIG. 7 shows electrochemiluminescence (ECL) values for high positive control HPC (1000 ng/mL). ECL values are plotted against different test human plasmas.

FIG. 8 shows electrochemiluminescence (ECL) values for IgG coat controls. The ECL values were observed over twenty-four runs.

FIG. 9 shows electrochemiluminescence (ECL) values for IgM coat controls. The ECL values were observed over twenty-four runs.

FIG. 10. shows screening sensitivity and confirmatory sensitivity assay results.

FIG. 11 summarizes assay results demonstrating screening drug tolerance and confirmatory drug tolerance.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

### Abbreviations

ADA Anti-Drug Antibody

C1INH C1 Esterase Inhibitor, or C1-inhibitor

CI Confidence Interval

CCP Confirmation Assay Cutpoint

CF Cut point Factor

CV C Coefficient of variation

df Degrees of freedom

ECL Electrochemiluminescence

F Fail

FPER False-Positive Error Rate

hIgG Human IgG

hIgM Human IgM

HPC High positive control

LPC Low positive control

Max Maximum

Min Minimum

MRD Minimum required dilution

MSD MesoScale Discovery

n Number of samples

NA Not applicable

NC Negative control

NF Normalization Factor

P Pass

pAb Polyclonal antibody

PC Positive Control

PSCP Plate specific cut point

SCP Screening Assay Cutpoint

shIgG Sheep IgG

SD Standard deviation

Immunogenicity of drug products, particularly protein drug products, leading to development of ADAs can be a problematic in drug treatment protocols because of the potential serious side effects and reduction in drug efficacy resulting from such immunogenicity. Development of ADAs can also result in uncertainty in interpretation of clinical and pre-clinical data related to toxicity, pharmacokinetic and pharmacodynamics.

For a drug found in high circulating concentrations, any circulating ADAs are typically bound to the circulating drug (drug interference) making the ADA unavailable for detection. Accordingly, development of drug tolerant immunogenicity assays present challenges in detection of ADAs. The following disclosure provides an immunoassay based on acid dissociation of drug-ADA complexes within a sample; neutralization of the sample simultaneous with or closely followed by contact with affinity-labeled drug; capture of ADA-affinity labeled drug complexes on a an affinity binding substrate; and detection of captured complexes via use of tagged anti-human immunoglobulin secondary antibodies.

The immunoassay comprises the steps of (i) providing a test sample with an acidic environment for dissociation of anti-drug antibodies from the drug within a test sample; (ii) neutralization of the test sample containing the dissociated drug-ADA complexes (iii) incubation of the sample with excess affinity-labeled drug; (iv) capture of the resulting ADA-affinity labeled drug complexes on an affinity binding substrate surface; (v) addition of tagged anti-human immunoglobulin secondary antibodies that bind to the captured ADA-affinity labeled drug complexes; and (vi) quantification of captured antibody-drug-ADA complexes.

In one aspect, the present disclosure relates to an anti-drug antibody immunoassay for detection of the presence of anti-C1-Inhibitor antibodies (C1INH-ADA) in a sample. The assay comprises the steps of (i) adding an acid solution to a test sample for dissociation of C1INH-ADA complexes in the test sample; (ii) neutralization of the test sample containing the dissociated C1INH-ADA; (iii) incubation of the test sample with excess affinity labeled C1INH resulting in formation of ADA-affinity labeled C1INH complexes; (iv) contact of the test sample with an affinity functionalized binding substrate surface resulting in capture of the ADA-affinity labeled C1INH complexes; (vi) addition of tagged anti-human immunoglobulin secondary antibodies that bind to the captured ADA-affinity labeled C1INH complexes; and quantification of captured ADA-affinity-labeled C1INH complexes.

The present disclosure relates more specifically to an anti-drug antibody immunoassay for detection of the presence of anti-C1-Inhibitor antibodies (C1INH-ADA) in a sample. The assay comprises the steps of (i) adding an acid solution to a test sample for dissociation of C1INH-ADA complexes in the test sample; (ii) neutralization of the test sample containing the dissociated C1INH-ADA; (iii) incubation of the test sample with excess biotinylated C1INH resulting in formation of ADA-biotinylated C1INH complexes; (iv) contact of the test sample with a streptavidin functionalized surface resulting in capture of the ADA-biotinylated C1INH complexes; (vi) addition of tagged anti-human secondary antibodies that bind to the streptavidin captured ADA-biotinylated C1INH complexes; and quantification of captured antibody-C1INH-ADA complexes.

The disclosure provides immunoassays for detection of anti-drug antibodies with specificity towards a wide variety of different drug products. In certain aspects, the drug products are protein-based products that have been developed to treat a wide variety of clinical indications, including cancers, autoimmunity/inflammation, exposure to infectious agents, and genetic disorders. Such therapeutic proteins include, for example, antibodies (including antibody fragments and fusion proteins), coagulation factors, hormones, growth factors, cytokines, enzymes and plasma proteins to name a few. In addition, the immunoassays may be used to detect anti-drug antibodies against nucleic acid-based drugs including RNA and DNA based drug products.

A test sample to be measured refers to a sample possibly containing drug-ADA complexes and, for example, is a sample collected from a subject being treated with the drug. The term “drug” as used herein refers to a chemical substance, including for example proteins or peptides, that are used to treat, cure, prevent, or diagnose a disease or to promote well-being in a treated subject. In a specific aspect, the sample may possibly contain C1INH-ADA complexes and, for example, is a sample collected from a subject for which C1INH is being administered. In other embodiments, the sample is obtained from a subject who has not recently been exposed to the drug, e.g., C1INH, or obtained from the subject prior to the planned administration of the drug. A subject may be a mammal, for example a human, with a disease or suspected of having a disease for which drug treatment is being, or is to be, administered. However, in some instances, the term “subject”, as used herein, refers to laboratory animal of an animal model study.

The term “sample” includes any biological specimen obtained from a subject. In some embodiments, the sample is derived from a bodily fluid or body tissue. A test sample may comprise a material selected from the group consisting of body fluids, blood, whole blood, plasma, serum, mucus secretions, saliva, tears, fine needle aspirate, lymph fluid or an immunoglobulin-enriched fraction derived from one or more of these tissues. One skilled in the art will appreciate that samples such as serum samples can be diluted prior to the analysis.

The immunoassay provided herein comprises a step wherein the sample to be tested is exposed to an acid solution for dissociation of the drug-ADA complexes found within the sample. In particular, a subject's sample can be incubated with an amount of acid that is sufficient to provide for the measurement of the presence or level of drug-ADA complexes. In a specific embodiment, exposure to an acid solution results in dissociation of C1INH-ADA complexes within the sample.

The acid solution to be utilized may be any acid solution that results in dissociation of the complexes within the sample. The amount of acid solution to be utilized is an amount that provides an acid environment sufficient to result in dissociation of the drug-ADA complex of interest and can be determined by one of skill in the art. Typically, such an acidic environment is in the pH range of pH 1.0 to pH 5.0, preferably pH 2.0 to pH 3.0, more preferably pH 2.6. For the assays disclosed herein, the test sample is contacted with an acid solution at a concentration of between about 0.1 M to about 1 M, more preferably 0.3 M. The acid solution can comprise an organic acid, an inorganic acid, or a mixture thereof. In some aspects, the acid solution comprises an acid selected from the group consisting of citric acid, glutamic acid, acetic acid, glycine/HCl and any combinations thereof. In an exemplary embodiment, the acid solution comprises acetic acid. In embodiments, the sample is contacted with an acid for an amount of time that is sufficient to dissociate drug-ADA complexes. Additional methods, well known to those skilled in the art, for dissociation of drug-ADA complexes may also be used. For example, dissociation may be achieved through application of heat, with or without EDTA.

Following the acidic dissociation, the sample is neutralized and labeled drug, e.g. labeled C1INH is added. The step of neutralizing the acid comprises raising the pH of the sample to allow the formation of a complex between the labeled drug and ADAs as described herein. In some embodiments, the acid is neutralized by the addition of one or more neutralizing agents such as, for example, strong bases, weak bases, buffer solutions, and combinations thereof. One skilled in the art will appreciate that neutralization reactions do not necessarily require a resultant pH of 7 but rather a pH that allows the formation of labeled drug/ADA complexes.

In certain aspects, affinity-binding pairs comprising a first member of a binding pair and a second member of a binding pair are used for capture of drug-ADA complexes on an affinity binding substrate surface. In such affinity binding pairs, the first member of the binding pair has binding affinity for the second member of a binding pair. Such affinity binding pairs for use in the methods provided herein include, for example, biotin/streptavidin, biotin/avidin, biotin/neutravidin, biotin/captavidin, epitope/antibody, protein A/immunoglobulin, protein G/immunoglobulin, protein L/immunoglobulin, GST/glutathione, His-tag/Nickel, antigen/antibody, FLAG/M1 antibody, maltose binding protein/maltose, calmodulin binding protein/calmodulin, enzyme-enzyme substrate, and receptor-ligand binding pairs. In a specific embodiment, the affinity binding pair comprises a biotin/streptavidin binding pair.

In one aspect, the excess drug to be added to the test sample is labeled with a first member of the binding pair and the binding substrate surface comprises the cognate second member of the binding pair. In a specific embodiment, the affinity binding pair comprises a biotin/streptavidin binding pair. In such an instance, the drug, e.g., C1INH is labeled with biotin and the binding substrate surface comprises streptavidin molecules. A binding substrate surface may be a tube, cuvette, microtiter plate, beads or microparticles. Such substrates include, but are not limited to, those made of polystyrene, polycarbonate, polyvinyltoluene, polypropylene, polyethylene, polyvinyl chloride, nylon, polymethacrylate, latex, gelatin, agarose, cellulose, sepharose, glass, metal, ceramic, a magnetic substance, or the like.

In one aspect, the streptavidin surface is a streptavidin coated microtiter plate. In one aspect, the binding substrate surface is Meso Scale Discovery (MSD)-Gold streptavidin-coated plates.

The immunoassay disclosed herein, comprises the step of detecting captured ADA-binding affinity labeled drug complexes. In embodiments, any directly or indirectly labeled reagent that binds to the captured ADA-binding affinity labeled drug complexes may be used. In an embodiment tagged anti-human antibodies may be used in the practice of the assay for detection of ADA-binding affinity labeled drug complexes. The tagged anti-human immunoglobulin antibodies include, polyclonal, monoclonal and fragments of antibodies that recognize and bind to human antibodies. Such anti-human antibodies include anti-human antibodies tagged with a detectable label. In addition, aptamers, such as oligonucleotide or peptide molecules that bind to a specific target molecule, may be used.

The detectable label may comprise, for example, a label selected from the group consisting of a hapten, radioactive isotope, an enzyme, a fluorescent label, a chemiluminescent label, and electro-chemiluminescent label. Methods for coupling detection reagents such as antibodies, e.g., anti-human antibodies, to detectable labels are well known in the art, as are methods for imaging using detectable labels. Such labeled reagents may employ a wide variety of labels. Detection of the formation of captured ADA-binding affinity labeled drug complexes can be facilitated by attaching a detectable substance to the detection reagent, such as an anti-human antibody. Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like.

Labeled antibodies can be tagged with such labels by known methods. For instance, coupling agents such as aldehydes, carbodiimides, dimaleimide, imidates, succinimides, bid-diazotized benzadine and the like are used to tag the antibodies with the above-described fluorescent, chemiluminescent, and enzyme labels. An enzyme is typically combined with an antibody using bridging molecules such as carbodiimides, periodate, diisocyanates, glutaraldehyde and the like. Various labeling techniques are described in Morrison, Methods in Enzymology 32b, 103 (1974), Syvanen et al., J. Biol. Chem. 284, 3762 (1973) and Bolton and Hunter, Biochem J. 133, 529(1973).

In an embodiment, the anti-human antibodies are labeled with an electrochemiluminescence moiety. Electrochemiluminescent labels generate light when stimulated by electricity in the appropriate chemical environment. In one embodiment, the detectable label comprises an electrochemiluminescent label comprising a sulfo-TAG® label and allows for ultra-sensitive detection. In such an instance, the sulfo-TAG labeled antibodies are used in conjunction with a binding substrate surface comprising Meso Scale Discovery (MSD)-Gold streptavidin-coated plates. Electricity is applied to the plate electrodes by an MSD instrument leading to light emission by SULFO-TAG labels. Light intensity is then measured to quantify ADAs present in the test sample.

Methodologies and techniques for performing the above ADA assays, such as assay conditions, assay buffers, washing steps, solid supports, suitable tags/labels and methods for linking them to the detection agent, techniques for detecting/measuring the detectable label, and equipment for performing the assays are known to those skilled in the art.

The present disclosure further provides kits that are assembled for determining the presence or absence of ADAs in a test sample. The kits may comprise control samples and/or instructions and, in a container, reagents including (i) for contacting the sample with an acid solution; (ii) for neutralization of the sample. The kit will further comprise one or more of the following reagents (i) a binding affinity labeled drug; (ii) an affinity binding substrate for capture of ADA-binding affinity labeled drug complexes; and tagged anti-human antibodies.

### Example

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be effected by one skilled in the art without departing from the scope or spirit of this disclosure. Additionally, the elements and features shown or described in connection with certain embodiments may be combined with the elements and features of certain other embodiments without departing from the scope of this disclosure, and that such modifications and variations are included within the scope of this disclosure. Accordingly, the subject matter of this disclosure is not limited by what has been particularly shown and described.

Hereditary angioedema (HAE) is a rare disorder that leads to swelling consequent to excess bradykinin generation. When this occurs in the airways, attacks can be life-threatening. Excess bradykinin generation results from a deficiency of C1-Inhibitor (C1INH), a serpin important in control of plasma serine proteases that release bradykinin from high molecular weight kininogen. Clinical trials of several protein replacement therapeutics have reported low incidence of anti-drug antibodies (ADA) to C1INH in subjects; however, the drug tolerance of these assays has not been described. High levels of soluble analyte are known for interference in ADA assays; normal serum concentrations of C1INH range from 180-200 μg/mL (1.7-2 μM). Accordingly, there is a need to develop and validate a well-controlled and sensitive assay for C1INH immunogenicity. Described below is a highly drug-tolerant assay for ADA to C1INH in human serum.

Specifically, a dual secondary antibody-based ADA assay (based on Affinity Capture Elution without the need for two solid phases) for human anti-C1INH antibody is disclosed. As described in detail below, C1INH-ADA complexes in undiluted serum samples were acid-dissociated. Released antibody was neutralized and incubated with excess biotinylated C1INH. The resulting ADA-biotinylated C1INH complexes were captured on Meso Scale Discovery (MSD)-Gold streptavidin-coated plates. Sulfo-tagged anti-human (h)IgG was allowed to bind to captured complexes and quantitated using standard MSD protocols. Sample dilution after acidification and neutralization was 20-fold. Since human anti-hC1INH is unavailable, sheep anti-hC1INH (PC) served as surrogate positive control. Detector reagent concentrations (analyte-specific anti-hIgG/IgM and PC-specific anti-sheep IgG) were optimized to ensure equivalent sensitivities using control wells incubated with biotinylated hIgG or hIgM.

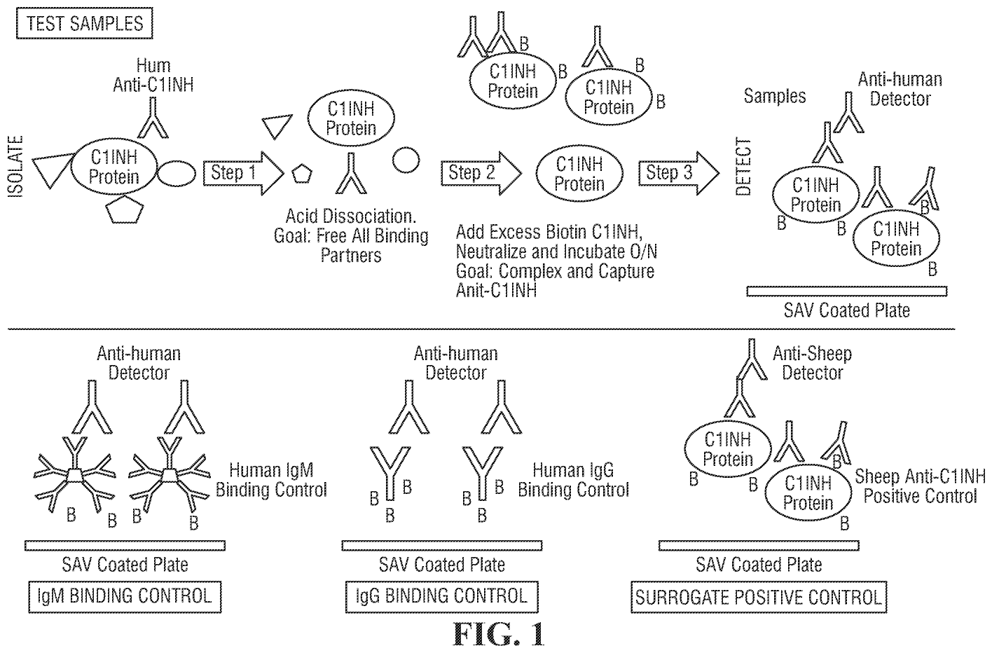

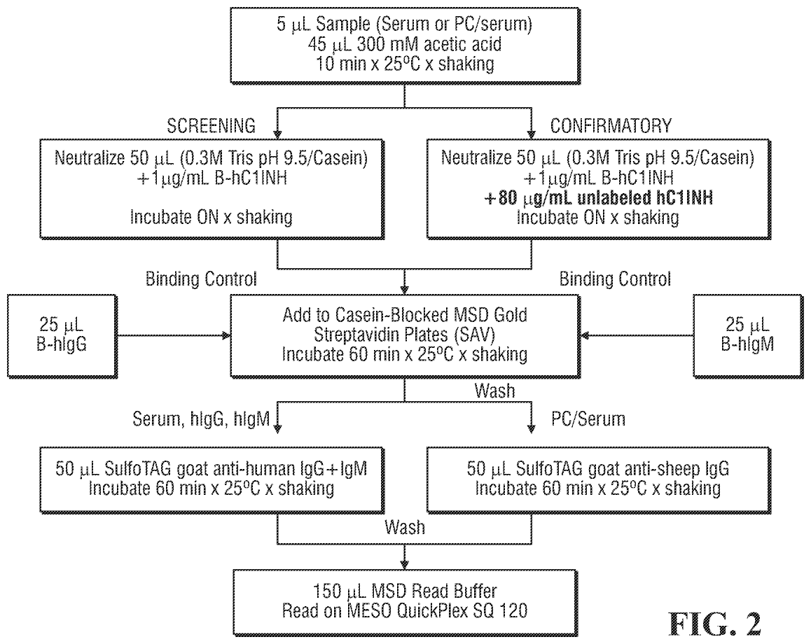

A dual secondary antibody-based ADA assay (based on Affinity Capture Elution without the need for two solid phases) for human anti-C1INH antibody was developed (see, FIG. 1 and FIG. 2) C1NH-ADA complexes in undiluted plasma samples were acid-dissociated. Released antibody was neutralized and incubated with excess biotinylated C1INH. The resulting ADA-biotinylated C1INH complexes were captured on Meso Scale Discovery (MSD)-Gold streptavidin-coated plates. Sulfo-tagged anti-human (h) IgG was allowed to bind to captured complexes and quantitated using standard MSD protocols. Sample dilution after acidification and neutralization was 20-fold. Since human anti-hC1INH is unavailable, sheep anti-hC1INH (PC) served as surrogate positive control. Detector reagent concentrations (analyte-specific anti-hIgG/IgM and PC-specific anti-sheep IgG) were optimized to ensure equivalent sensitivities using control wells incubated with biotinylated hIgG or hIgM. Specific details of the utilized materials and methods are described below.

Samples and QC were removed from the freezer and placed at RT to thaw. A 0.742 mg/mL stock of biotinylated C1-INH was diluted to 1.0 μg/mL in neutralization buffer (30% 1M Tris HCL pH 9.5 in blocker Casein in TBS) (“labeled neutralization buffer”). 5.0 μL of each sample and QC were transferred to a dilution plate. 45.0 μL 300 mM acetic acid was added to each well of the dilution plate containing a sample or QC. Next, samples were incubated for 10 minutes at 25±2° C. on setting 2 of Labline Titre Plate Shaker or 100 rpm to allow C1-INH-ADA dissociation. 50.0 μl labeled neutralization buffer was then added to each well of the dilution plate containing the acidified sample and QC. Incubation was done for 16-20 hours at 25±2° C. on setting 2 of Labline Titre Plate Shaker or 100 rpm.

MSD SAV plates and buffers were placed at RT for at least 15 minutes prior to use. Blocking of MSD plates was done as follows: 150 μl of blocker casein in TBS was added to each well and incubation was for 1-hour±10 minutes on 25±2° C. on (setting 0).

Preparation of Human Detection Controls (volume for two plates) was as follows. To prepare a 10.0 μg/ml intermediate solution, a 1.00 mg/mL stock Biotin-Tagged-Human IgG was diluted to 10.0 μg/mL in Blocker Casein in TBS. The 10.0 μg/ml intermediate biotin-tagged-human IgG was diluted to the working concentration of 100 ng/mL in Blocker Casein in TBS. To prepare a 10.0 μg/ml intermediate solution, a 1.00 mg/mL stock Biotin-Tagged-Human IgM was diluted to 10.0 μg/mL in Blocker Casein in TBS. The 10.0 μg/ml intermediate biotin-tagged-human IgM was diluted to the working concentration of 100 ng/mL in Blocker Casein in TBS.

The blocked assay plate was washed 3×, on a plate washer, with ELISA Wash Buffer (0.05% Tween 20 in 1×PBS) by adding 300 μL of buffer to each well. The blocked assay plate was inverted and tapped on absorbent paper after the final wash. Neutralized samples and QC were removed from the shaker.

Sample Step was as follows. 25.0 μL of each neutralized QC and/or sample, 100 ng/mL Biotin tagged IgG and Biotin tagged IgM were transferred to the respective wells of assay plate per plate map was done. Incubation was done for 1-hour±10 minutes on 25±2° C. Jitterbug with shaking (setting 0).

Detection Preparation was as follows. The 500 μg/mL stock Sulfo-tagged-anti-sheep AB detection antibody was diluted to 500 ng/mL in Blocker Casein in TBS. The 1.62 mg/mL stock Sulfo-tagged-Fab anti-hu-IgG+IgM detection antibody was diluted to 16.2 μg/mL. The 16.2 μg/mL Intermediate sulfo-tag-Fab-anti-hu-IgG+IgM detection antibody was diluted to 162 ng/mL in Blocker Casein TBS. The 162 ng/mL Intermediate sulfo-tag-Fab-anti-hu-IgG+IgM detection antibody was diluted to 1.0 ng/mL in Blocker Casein TBS.

The assay plate was washed 3× on a plate washer with ELISA Wash Buffer by adding 300 μL of buffer to each well. The plate was then inverted and tapped on absorbent paper after the final wash.

The detection step was as follows. 50.0 μL of each detection antibody was added to the respective wells per plate map. Incubation was carried out for 1 hour±10 minutes on 25±2° C. Jitterbug with shaking (setting 0).

The assay plate was then washed 3× on a plate washer with ELISA Wash Buffer by adding 300 μL of buffer to each well. The plate was inverted and tapped on absorbent paper after the final wash. The stop step was performed by addition of 150 μL of MSD Read Buffer to each well. The plate was then read.

The Confirmation Assay procedure was conducted as follows: C1-INH (Stock at 0.5 mg/mL) was diluted to 80.0 μg/mL in labeled Neutralization Buffer. 50 μL of drug spiked labeled neutralization buffer was added per well to dilution plate containing acid treated samples according to plate map. Incubation was conducted 16-20 hours at 25±2° on setting 2 of LablineTitre Plate Shaker or 100 rpm. The screening assay was then performed as described above.

The cut point determination was conducted using normal plasma samples obtained from 30 individual male and female humans. Each of the samples was run at the MRD of 1/20 (unspiked) and at the MRD in the presence of 80 μg/mL human C1INH (spiked). Two operators each ran all 30 samples spiked and unspiked, in two independent runs, for a total of four runs.

Uncorrected ECL values from unspiked samples were used to define the plate-specific cut point factor. Percent inhibition by spiked drug was calculated for each sample and used to define the % inhibition cut point. To identify the statistical outliers, the “outlier box-plot criteria” was used. More precisely, all samples in an individual run above Q3+1.5*(Q3−Q1) or below Q1−1.5*(Q3−Q1), where Q3 and Q1 represented respectively the 75th and 25th percentiles, were considered as Analytical outliers. Following removal of analytical outliers, all samples above Q3+1.5*(Q3−Q1) or below Q1−1.5*(Q3−Q1), where Q3 and Q1 represented respectively the 75th and 25th percentiles, on averaged (mean) data, were considered as Biological outliers and all such were removed from the data. The outliers were evaluated using ECL values for CP and percentage inhibitions for CCP.

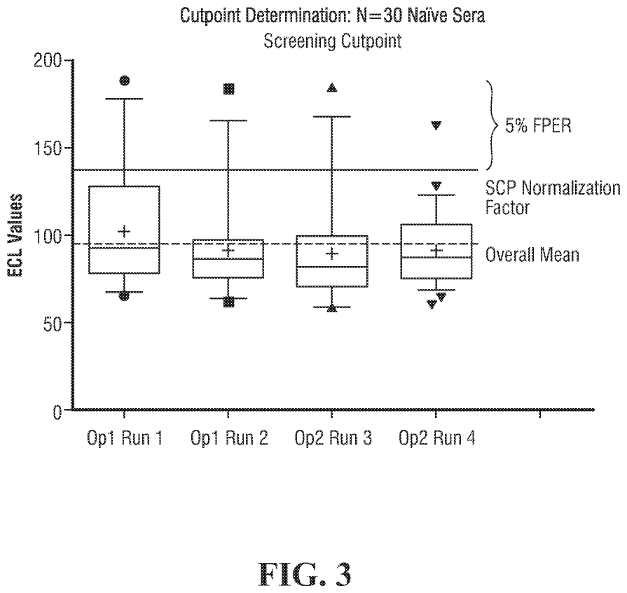

Following elimination of the outliers (analytical and biological) and prior to the evaluation of the CP, the distribution normality of the original data was tested via Shapiro-Wilk's test to decide which scale (original or log) to use for the CP determination. For the screen and titer assays, the result of this test was found to be significant at the 10% level (Shapiro-Wilk W value is greater than the P value). Therefore, the nonparametric method was used on the log-transformed data and the screening cut point was determined as the antilog of the 95th percentile value. The screening cut point factor (NF) of 1.457 was determined as the average (CPrun/Median NCrun) from the four runs. (FIG. 3)

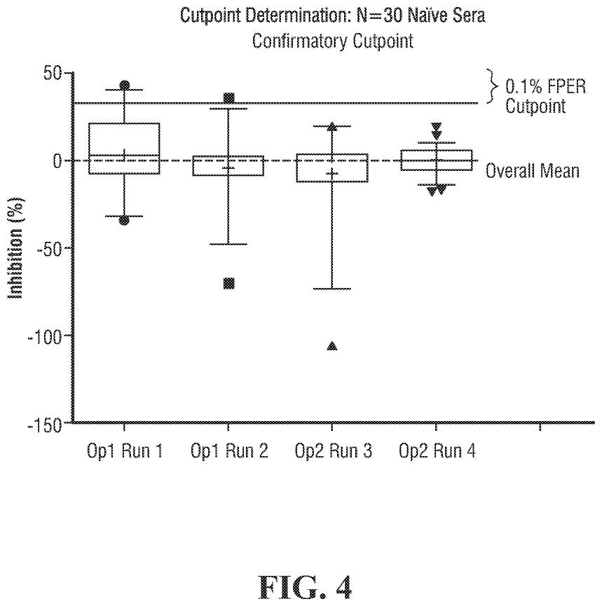

The data with outliers removed was used for the calculation of the CCP and was evaluated by Shapiro-Wilk's test for each run and was evaluated using the ratio of (s/us) where “s” denotes spiked and “us” denotes unspiked values. If the result of this test was not found to be significant at the 10% level and the distribution was found to be symmetrical, then the parametric method was used to calculate the CCP. More precisely, the CCP was defined as mean (percentage inhibition)+3.09*SD (percentage inhibition). This is the case for runs 1 and 3. Runs 2 and 4 were found to be non-normally distributed and therefore the nonparametric method was used on the log-transformed data and the confirmatory cut point was determined as the antilog of the 99.9th percentile value. The average cut point from the four runs is 34.0% Inhibition. (FIG. 4)

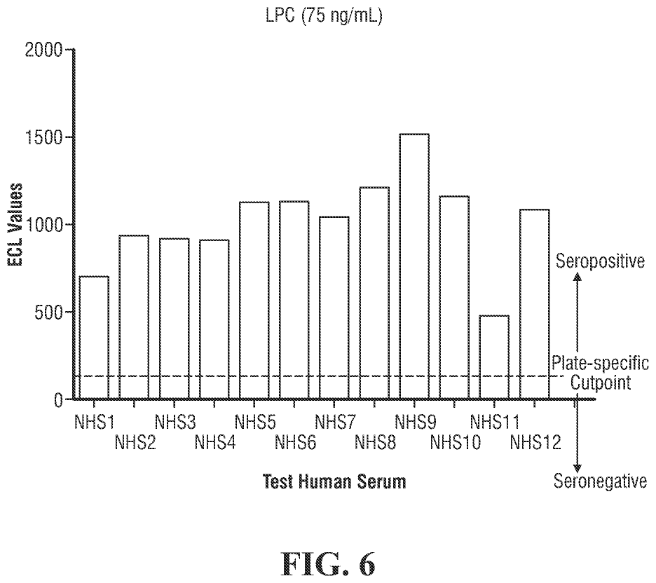

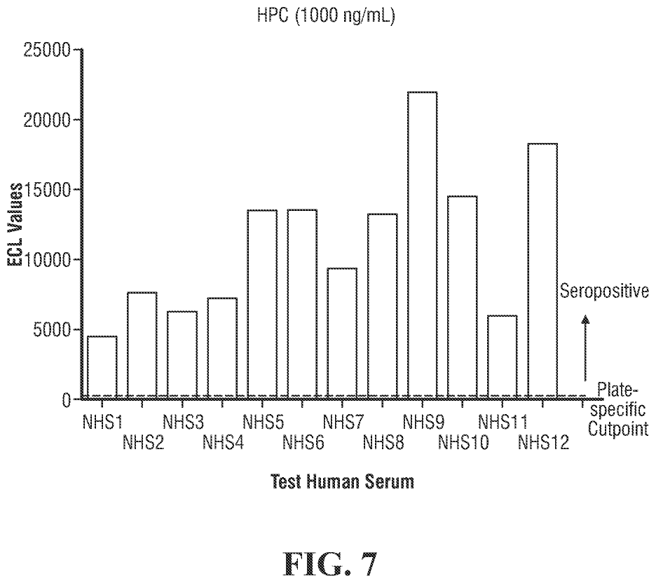

Precision evaluation (% CV) of the ECL values obtained in the screening assay for the HPC (1000 ng/mL) and LPC (75 ng/mL) were <20% CV (20.3 and 15.8%, respectively) over 6 runs by two analysts. (FIG. 6 and FIG. 7) To assess the precision of the assay at lower ranges, a second study was performed with HPC (1000 ng/mL). LPC (75 ng/mL), LPC1 (50 ng/mL), LPC2 (25 ng/mL) and LPC3 (12.5 ng/mL). The data demonstrate precision of ≤25% CV for all levels of PC and for the NC.

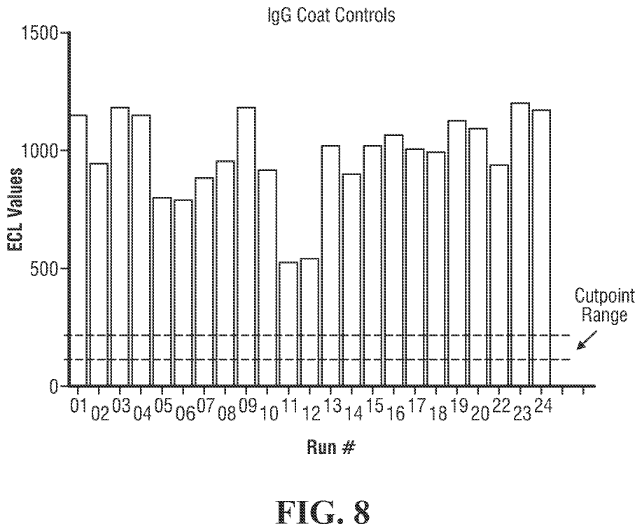

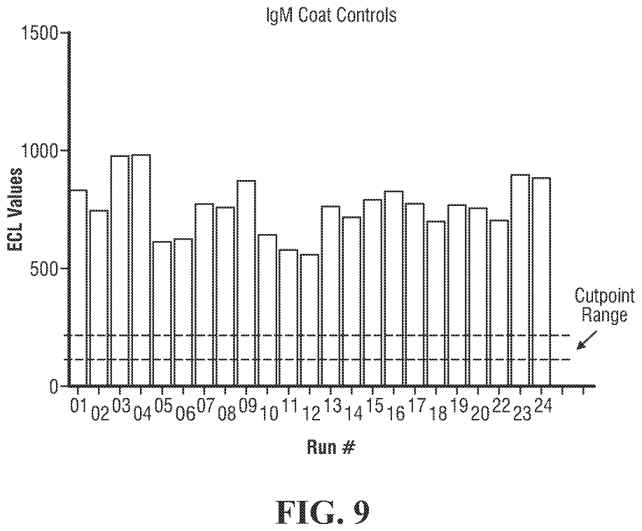

Precision of the human IgG and human IgM coating controls ECL signal was also <20%. (FIG. 8 and FIG. 9)

Precision of the % Inhibition values calculated for the HPC and LPC in six runs by two analysts was 1.5 and 7.7% CV, respectively. An additional precision study with lower PC concentrations also maintained precision of <20% CV.

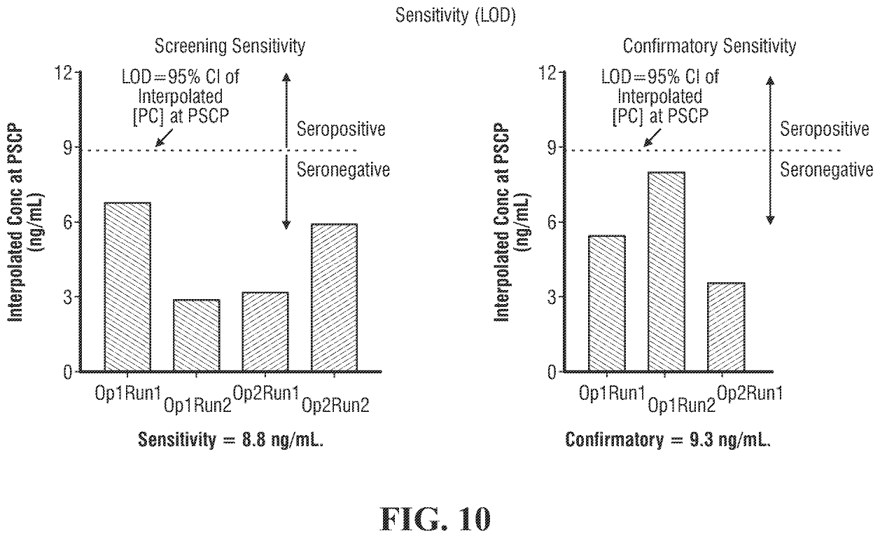

The sensitivity of the screening assay was determined using the surrogate positive control, sheep anti-C1INH pAb, spiked into normal human plasma at concentrations ranging from 312.5-2.4 ng/mL (before application of the MRD of 20) prepared four independent times and analyzed in two runs each by two different analysts. The concentration associated with the cut point was determined and the sensitivity defined as the 95^{th} C.I. The data indicate the method sensitivity is 8.8 ng/mL. (FIG. 10)

The sensitivity of the confirmatory assay was determined using the surrogate positive control, sheep anti-C1INH pAb, spiked into normal human plasma at concentrations ranging from 31.3-0.24 ng/mL (before application of the MRD of 20) prepared four independent times and analyzed in two runs each by two different analysts. The concentration associated with the cut point was determined and the sensitivity defined as the 95^{th} C.I. The data indicate the method sensitivity is 9.3 ng/mL (FIG. 10).

It is important to note that due to potential affinity and avidity differences between the pAb used in this study and those of actual subjects, the antibodies used for this study do not fully represent the antibody repertoire of preclinical or clinical samples, and therefore the actual sensitivity value may vary for preclinical or clinical samples.

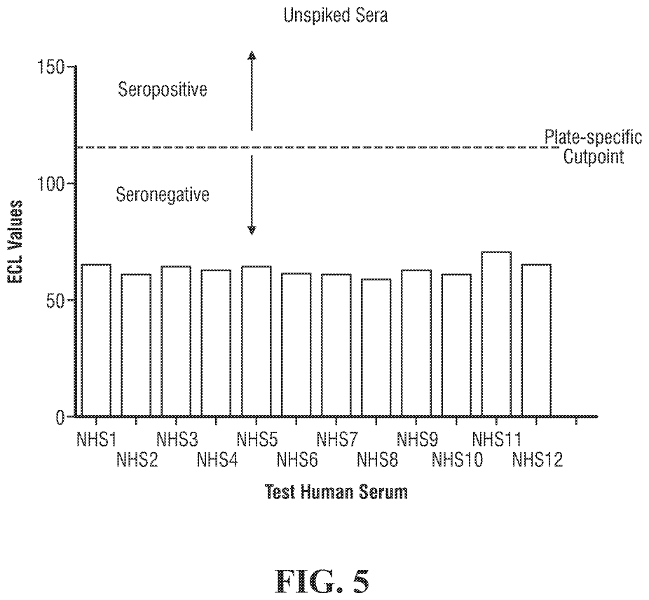

The screening assay method was demonstrated to be selective to the detection of low (75 ng/mL) and high (1000 ng/mL) levels of anti-drug (C1INH) antibodies. In this evaluation, individual normal plasma samples (n=12) spiked with polyclonal anti-hC1INH antibody scored positive in the screening assay and confirmed positive in the confirmatory assay. Unspiked controls scored negative. (FIG. 5)

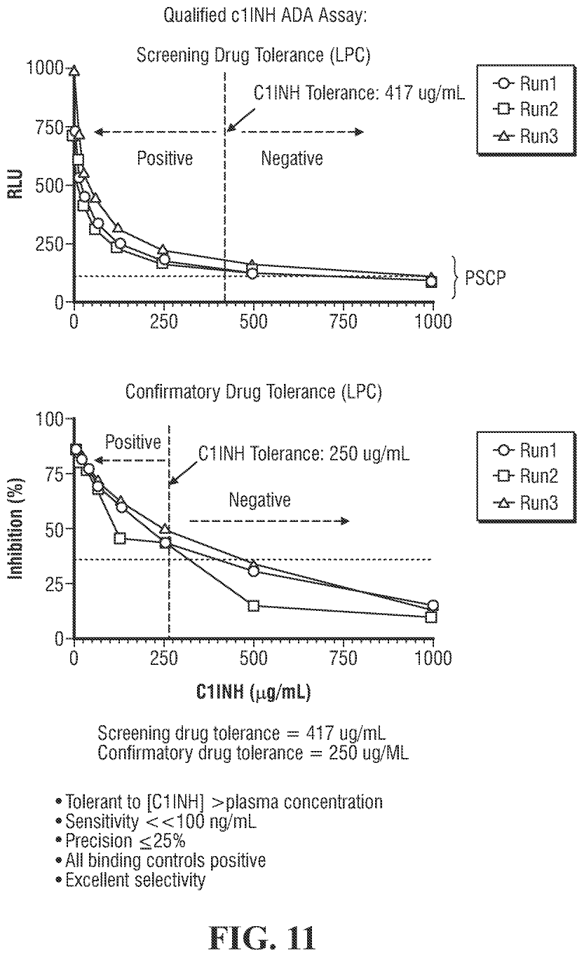

The ability of the assay to detect anti-hC1INH antibodies when hC1INH is present (drug tolerance) was evaluated by spiking plasma samples containing known concentrations of anti-hC1INH (HPC and LPC) with hC1INH (1000 ng/mL to 15.6 ng/mL). Drug tolerance is defined as the greatest amount of hC1INH present in a sample that still allows the sample to score positive for antibody. The screening assay is tolerant to 417 μg/mL of C1INH at 75 ng/mL level of antibody. The confirmatory assay is tolerant to 250 μg/mL of C1INH at the 75 ng/mL level of antibody. As expected, increasing levels of anti-drug antibody show increased tolerance to circulating C1INH. FIG. 11 demonstrates the conclusions determined for the disclosed qualified c1INH ADA assay. Below is a table representing the qualification parameters.

The qualified method complies with the 2019 FDA Guidance-required screening sensitivity of ≤100 ng/mL ADA in the presence of normal plasma levels of C1INH with acceptable intra- and inter-assay precision. The assay is therefore acceptably drug-tolerant and has acceptable precision and selectivity.

| Parameter | Result | |
|---|---|---|
| PC Precision (% CV) | LPC (75 ng/mL): | 16% w/o Drug |
| | | 22% with 80,000 ng/mL Drug |
| | HPC (1000 ng/mL): | 20% w/o Drug |
| | | 25% with 80,000 ng/mL Drug |
| Sensitivity (LOD) | Screening assay: | 8.8 ng/mL |
| (surrogate PC sheep α-hClINH) | Confirmation assay: | 9.3 ng/mL |
| Selectivity in Human Serum | Unspiked matrix lots: | 100% negative |
| (12 Matrix Lots) | HPC and LPC matrix lots: | 100% positive |
| SCP Factor | 1.457 (non-parametric, 5% FPER) | |
| CCP | 34% Inhibition (0.1% FPER) | |
| Drug Tolerance for hClINH at LPC | Screening assay: | 417 μg/mL |
| | Confirmatory assay: | 250 μg/mL |

| | |
|---|---|
| IgG and IgM Binding Controls | All > PSCP |

What is claimed:

1. An immunoassay for detection of anti-drug antibodies within a test sample comprising the steps of (i) providing a test sample with an acidic environment for dissociation of anti-drug antibodies from the drug within a test sample; (ii) neutralization of the test sample containing the dissociated drug-ADA complexes (iii) incubation of the sample with excess binding affinity-labeled drug; (iv) capture of the resulting ADA-affinity labeled drug complexes on an affinity binding substrate surface; (v) addition of tagged anti-human antibodies; and (vi) quantification of captured antibody-drug-ADA complexes.

2. The immunoassay of claim 1, wherein the test sample is derived from a subject being treated with the drug, a subject who has not recently been exposed to the drug, or a subject prior to the planned administration of the drug.

3. The immunoassay of claim 2, wherein the subject is a mammal.

4. The immunoassay of claim 2, wherein the test sample comprises body fluids, blood, whole blood, plasma, serum, mucus secretions, saliva, lymph fluid or an immunoglobulin enriched fraction derived from one or more of these tissues.

5. The immunoassay of claim 1, wherein an affinity binding pair is used for capture of drug-ADA complexes on a substrate surface, wherein
the drug comprises a first member of a binding pair; and
the affinity binding substrate comprises a second member of a binding pair

6. The immunoassay of claim 5, wherein the binding pair is biotin/streptavidin.

7. The immunoassay of claim 5, wherein the affinity binding substrate surface is Meso Scale Discovery (MSD)-Gold streptavidin-coated plates.

8. The immunoassay of claim 1, wherein the anti-human antibodies are tagged with a detectable label selected from the group consisting of a phosphorescent moiety, luminescent moiety, electrochemiluminescent moiety, chromatic moiety, a radioactive isotope and an enzyme.

9. The immunoassay of claim 8, wherein the electrochemiluminescent moiety comprising a sulfo-TAG.

10. The immunoassay of claim 1, where in the acidic environment is provided by addition of an acid solution to a pH of about 2.6.

11. The immunoassay of claim 10, wherein the acidic environment is provided by addition of an acetic acid solution.

12. An immunoassay for detection of anti-CIINH antibodies within a test sample comprising the steps of (i) providing a test sample with an acidic environment for the dissociation of C1INH-ADA complexes within the test sample; (ii) neutralization of the test sample containing the dissociated C1INH-ADA complexes; (iii) incubation of the test sample with excess binding affinity-labeled C1INH resulting in formation of binding affinity-labeled C1INH-ADA complexes; (iv) capture of the resulting binding affinity-labeled C1INH-ADA complexes within the test sample on a functionalized affinity binding substrate surface; (vi) addition of tagged anti-human antibodies that bind to the captured binding affinity-labeled C1INH-ADA complexes; and (v) quantification of captured binding affinity-labeled C1INH-ADA complexes.

13. The immunoassay of claim 12, wherein the test sample is derived from a subject being treated with CIINH, a subject who has not recently been exposed to CIINH, or a subject prior to the planned administration of CIINH.

14. The immunoassay of claim 13, wherein the subject is a mammal.

15. The immunoassay of claim 14, wherein the test sample comprises body fluids, blood, whole blood, plasma, serum, mucus secretions, saliva, lymph fluid or an immunoglobulin enriched fraction derived from one or more of these tissues.

16. The immunoassay of claim 15, wherein an affinity binding pair is used for capture of CIINH-ADA complexes on a substrate surface, wherein
the CIINH comprises a first member of a binding pair; and
the affinity binding substrate comprises a second member of a binding pair

17. The immunoassay of claim 16, wherein the binding pair is biotin/streptavidin.

18. The immunoassay of claim 16, wherein the affinity binding substrate surface is Meso Scale Discovery (MSD)-Gold streptavidin-coated plates.

19. The immunoassay of claim 1, wherein the anti-human antibodies are tagged with a detectable label selected from the group consisting of a phosphorescent moiety, luminescent moiety, electrochemiluminescent moiety, chromatic moiety, a radioactive isotope and an enzyme.

20. The immunoassay of claim 9, wherein the electrochemiluminescent moiety comprising a sulfo-TAG.

21. The immunoassay of claim 1, where in the acid base environment is provided by addition of an acid solution to a pH of about 2.6.

22. The immunoassay of claim 11, wherein the acid base environment is provided by addition of an acetic acid solution.

23. A kit for determining the presence or absence of ADAs in a test sample said kit comprising (i) instructions and (ii) one or more reagents selected from the group consisting of an acid solution; a neutralization buffer, a binding affinity labeled drug; an affinity binding substrate for capture of ADA-binding affinity labeled drug complexes; and tagged anti-human antibodies.
